(19) United States
(12) Patent Application Publication
(10) Pub. No.: US 2024/0389934 A1
(43) Pub. Date: Nov. 28, 2024

(54) INTELLIGIBILITY ANALYSIS USING AUTOMATIC SPEECH RECOGNITION

(71) Applicant: International Business Machines Corporation, Armonk, NY (US)

(72) Inventors: Raquel Norel, Bronx, NY (US); Guillermo Cecchi, New York, NY (US); Bo Wen, New York, NY (US)

(21) Appl. No.: 18/321,841

(22) Filed: May 23, 2023

(51) Int. Cl. A61B 5/00; G10L 15/22; G10L 15/26; G10L 15/32; G10L 25/66

(52) U.S. Cl. CPC A61B 5/4803; G10L 15/22; G10L 15/26; G10L 15/32; G10L 25/66

(57) ABSTRACT

Intelligibility analysis of a speech recording is provided. A first transcript for the speech recording is generated using a first automatic speech recognition (ASR) model. A second transcript for the speech recording is provided. The first transcript and the second transcript are compared. An intelligibility score is determined based on the comparing the first transcript and the second transcript.

### BACKGROUND

The present disclosure relates to computer technology for medical evaluation, and more specifically, to automatic processing of patient speech data.

In speech communication, intelligibility is a measure of how comprehensible speech is in given conditions. Intelligibility can be used to monitor disease progression and quality of life, since communication affects quality of life. Intelligibility is traditionally assessed by highly trained professionals. This makes intelligibility analysis expensive, not scalable, and biased (i.e., not all professionals will give the same score to the same patient speech).

### SUMMARY

According to embodiments of the present disclosure, a method for intelligibility analysis of a speech recording is provided. The method includes providing the speech recording, generating a first transcript for the speech recording using a first automatic speech recognition (ASR) model, providing a second transcript for the speech recording, comparing the first transcript and the second transcript, and determining an intelligibility score based on the comparing the first transcript and the second transcript. Determining an intelligibility score based on a transcript generated by ASR transcription may allow for a more unbiased intelligibility analysis that results in greater consistency.

According to optional embodiments, the second transcript is generated using a second ASR model to the speech recording, wherein the second ASR model is more accurate than the first ASR model. Using two ASR models to generate the two transcripts may allow for intelligibility analysis that is less expensive, more scalable, and more consistent as the analysis may be done automatically without the need for manual transcription.

According to optional embodiments, the second transcript is a script associated with the speech recording. These embodiments may allow for intelligibility analysis that is less expensive and more scalable than embodiments that require manual transcription for the second transcript.

According to additional embodiments of the present disclosure, a system and a computer program product for performing the methods are provided.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts an example computing environment, according to embodiments.

FIG. 2 depicts a block diagram of an example intelligibility analysis module, according to embodiments.

FIG. 3 depicts a flow diagram of an example method for intelligibility analysis, according to embodiments.

FIG. 4 depicts an illustrative flow diagram of intelligibility analysis according to an embodiment of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to automated intelligibility analysis, and more particular aspects relate to intelligibility analysis using automatic speech recognition models. While the present disclosure is not necessarily limited to such applications, various aspects of the disclosure may be appreciated through a discussion of various examples using this context.

Because intelligibility is traditionally assessed by highly trained professionals, intelligibility analysis is traditionally expensive, not scalable, and biased (i.e., not all professionals will give the same score to the same patient speech). Providing intelligibility analysis in a less expensive and scalable manner may allow for better access to monitoring disease progression and quality of life for patients. Further, providing intelligibility analysis in an unbiased way may allow for greater consistency in intelligibility analysis and provide the opportunity to collect consistent data for research related to intelligibility and disease.

Embodiments of the present disclosure provide an automated method to analyze intelligibility of a person. A first transcript of a speech recording of the person is generated using an automatic speech recognition model (ASR). A second transcript for the speech recording is provided. The first transcript and the second transcript are compared, and an intelligibility score is determined based on the comparison. Determining an intelligibility score based on a transcript generated by ASR transcription may allow for a more unbiased intelligibility analysis that results in greater consistency.

ASR models can vary in their transcription accuracy. This variance can be based on, for example, the size of the model (e.g., the number of layers and parameters of a model) and the training of the model (e.g., type and volume of the training data). Some embodiments of the present disclosure may take advantage of the imperfections of less accurate models to evaluate intelligibility. For normal speech, less accurate models and more accurate models can both generate a highly accurate transcript. However, when a person's speech is less intelligible (e.g., because of a speech impediment), the transcripts produced by the less accurate model and the more accurate model diverge. This divergence can indicate the level of intelligibility of a person's speech.

Thus, in some embodiments, the second transcript is generated using a second ASR model, which is more accurate than the first ASR model, to the person's same speech. The second ASR model may process the speech recording at any time with respect to the first ASR model processing the speech recording. That is, the second ASR model may process the speech recording prior to, after, concurrently, or in a partially or wholly temporally overlapping manner with respect to the first ASR model processing the speech recording. The use of two ASR models to generate the two transcripts may allow for intelligibility analysis that is less expensive, more scalable, and more consistent as the analysis may be done automatically without the need for manual transcription.

In other embodiments, the second transcript may be a script that the person read from to provide their speech (e.g., the Bamboo passage). These embodiments may also allow for intelligibility analysis that is less expensive and more scalable. However, some of the accuracy in the intelligibility analysis relies on the person accurately reading the script. In other embodiments, a second transcript may be a manually-produced transcript provided by a person listening to the speech. These embodiments may be less expensive and more scalable than intelligibility analysis provided by a highly trained professional. However, it may be more expensive and less scalable than using a second transcript that is a script or is generated by a second ASR, because a person is required to manually transcribe the speech.

Any suitable similarity metric may be used in comparing the first transcript and the second transcript. Example similarity metrics include word error rate (WER) and match error rate (MER). WER is the proportion of word errors to words processed. MER is the probability of a given match being incorrect. In some embodiments, WER or MER may be computed using the second transcript (e.g., the script, manual transcription, or transcript generated by the more accurate ASR model) as the ground truth such that “errors” are determined with regards to the first transcript being different from the second transcript.

In some embodiments, the similarity metric may be converted to an intelligibility score using threshold values. Traditionally, intelligibility is rated on a five-point scale with level 1 being completely unintelligible and level 5 being completely intelligible. Thus, in some embodiments, the similarity metric may be converted to this five-point scale using threshold values. This may allow for automated intelligibility analysis to be comparable to intelligibility analysis performed manually by trained specialists. For example, intelligibility may be level 5 for a similarity metric less than value A, level 4 for a similarity metric between value A and value B, level 3 for a similarity metric between value B and value C, level 2 for a similarity metric between value C and value D, and level 1 for a similarity metric greater than value D. The five-point scale is just one example of an intelligibility score and is not meant to be limiting. Any suitable intelligibility score may be used. For example, an intelligibility scale with more or less levels may be used. In some embodiments, one or more mathematical operations may be performed on the similarity metric to convert the metric into an intelligibility score.

Any suitable ASR models may be used. ASR models may be implemented, for example, using artificial neural networks or hidden Markov models. As mentioned previously, in embodiments where a second ASR model is used to generate the second transcript, the second ASR model may be more accurate than the first ASR model. Whisper, created by Open AI, has ASR models of various sizes, ranging from “Tiny” to “Large” that vary in layers, width, heads, and parameters, with the larger models being more accurate. Thus, as an example where two ASR models are used, Whisper's Tiny model may be used to generate the first transcript and Whisper's Large model may be used to generate the second transcript.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as intelligibility analysis module 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

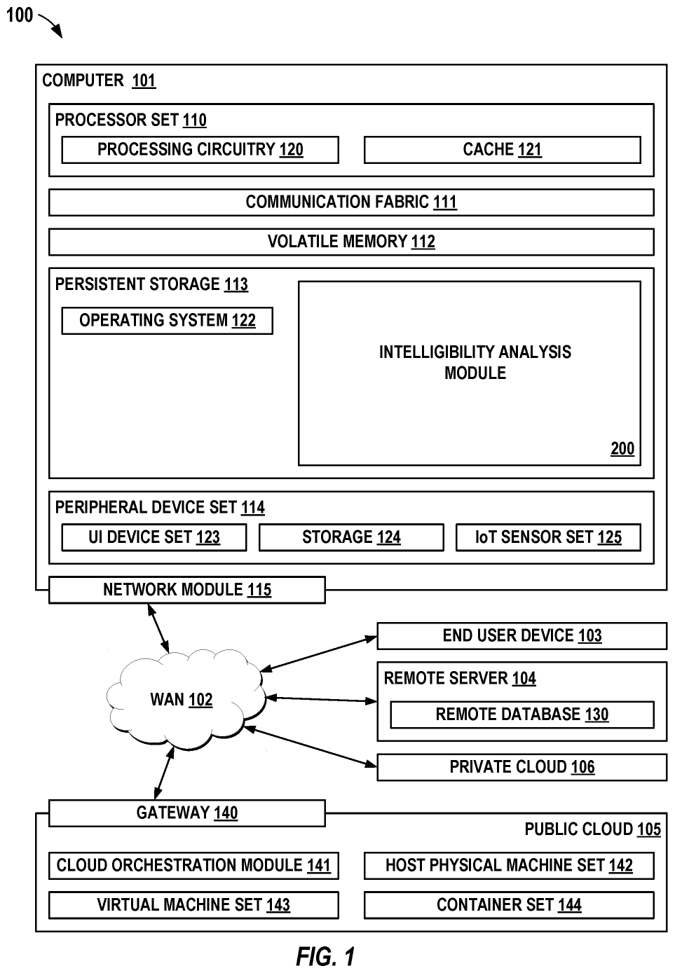

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

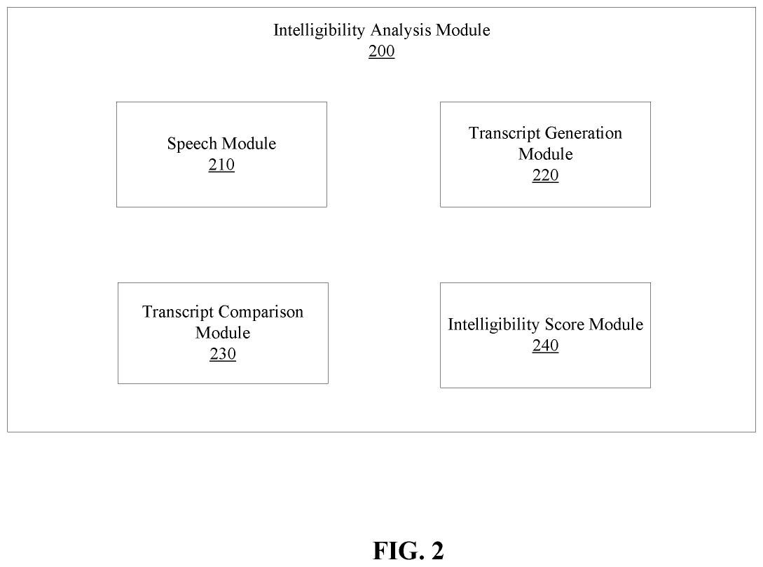

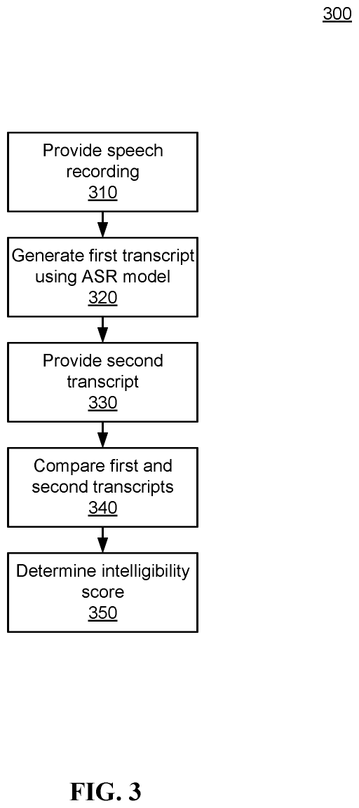

Referring now to FIG. 2, a block diagram of an example intelligibility analysis module 200 is depicted according to embodiments. Automated intelligibility module 200 may be configured to cause computer 101 to perform method 300 described in reference to FIG. 3. Intelligibility analysis module 200 may be configured to determine an intelligibility score for person's speech recording. As used herein, the term module includes hardware and/or software configured for implementing one or more logical functions. Intelligibility analysis module 200 includes speech module 210, transcript generation module 220, transcript comparison module 230, and intelligibility score module 240.

Speech module 210 may be configured to obtain a speech recording for a person and provide the speech recording to transcript module 220. In some embodiments, speech module 210 may be configured to capture the person's speech in real-time via a microphone communicatively coupled to a computing device. For example, speech module 210 may be configured to cause a microphone in UI device set 123 to capture audio of a person speaking. The recording of the person's speech may be stored in a local or remote storage. For example, the recording may be stored in volatile memory 112, persistent storage 113, storage 124, remote server 104, public cloud 105, or private cloud 106.

In some embodiments, speech module 210 may be configured to retrieve a speech recording that was previously recorded. For example, speech module 210 may be configured to retrieve the speech recording from remote storage such as remote server 104, public cloud 105, or private cloud 106.

Transcript generation module 220 may be configured generate one or more transcripts by inputting the speech recording provided by speech module 210 into one or more ASR models. The one or more ASR models may reside locally on computer 101 or remotely, such as on remote server 104, public cloud 105, or private cloud 106. When using multiple ASR models, the ASR models may reside on the same computing system or different computing systems.

Transcript comparison module 230 may be configured to compare two transcripts of a speech recording. Transcript comparison module 230 may be configured to calculate a similarity score between the two transcripts. In some embodiments, the two transcripts may be generated by different ASR models and provided by the transcript generation module 220.

In some embodiments, a first transcript may be generated by an ASR model and provided by the transcript generation module, and the second transcript may be a provided independently from an ASR model. In these embodiments, the second transcript may be stored with, or otherwise associated with, the speech recording. For example, the second transcript may be a script that the person was reading from when recording their speech or may be produced by manual transcription of the speech recording that has been stored in a storage communicatively coupled to computer 101. In some embodiments, transcript comparison module 230 may be configured to retrieve the second transcript from the remote or local storage. Alternatively, speech module 210 may be configured to retrieve the second transcript and provide it to transcript comparison module 230.

Intelligibility score module 240 may be configured to determine an intelligibility score based on the comparison between the two transcripts of the speech recording by transcript comparison module 230. In some embodiments, intelligibility score module 240 may be configured to determine the intelligibility score based on a similarity metric provided by transcript comparison module 230, as described herein.

Intelligibility analysis module 200 is just an example module. Other embodiments may contain more or less modules. In some embodiments, one or more of the logical functions of one module may be performed by a different module. While automated intelligibility module 200 is depicted as residing in computer 101, in some embodiments one or more of the modules that make up automated intelligibility module 200 may be located in different systems that are communicatively coupled.

Referring now to FIG. 3, a flow diagram of an example method 300 for intelligibility analysis is depicted, according to embodiments. Method 300 will be described herein as being performed by a computer system. For example, method 300 may be performed by computer 101 described in reference to FIG. 1.

At operation 310, the computer system provides a speech recording of a person. In some embodiments, the computer system may retrieve the speech recording from a storage device operatively coupled to the computer system. In some embodiments, the computer system may be operatively coupled to a microphone and may record the person's speech to a storage operatively coupled to the computer system. In some embodiments, the computer system may retrieve the speech recording via one or more networks from a cloud computing system or remote server.

At operation 320, the computer system generates a first transcript for the speech recording using an ASR model. The computer system may input the speech recording into the ASR model and obtain the first transcript as an output of the ASR model. In some embodiments, the computer system may contain the model and input the speech recording into the model to generate the first transcript. In some embodiments, the model may exist on a different computer system. For example, the computer system may communicate the speech recording to another computer system containing the model over one or more networks to process the speech recording and receive the transcript from the other computer system.

At operation 330, the computer system provides a second transcript for the speech recording. In some embodiments, the second transcript may be retrieved from a storage operatively coupled to the computer system. The second transcript may be a transcript stored by a user that generated by manual transcription or a script that was read by the person when their speech was recorded. In some embodiments, the computer system may generate the second transcript using a second ASR model. The second ASR model may be less accurate than the ASR model used to generate the first transcript. Similar to the ASR model used to generate the first transcript, the computer system may contain the second model and input the speech recording into the second model to generate the second transcript. In some embodiments, the second model may exist on a different computer system. For example, the computer system may communicate the speech recording to another computer system containing the model over one or more networks to process the speech recording and receive the transcript from the other computer system. The ASR models used to generate the first and second transcripts may exist on the same computer system or different computer systems.

At operation 340, the computer system compares the first and second transcripts. In some embodiments, the computer system may determine a similarity metric between the first and second transcripts. For example, the computer system may determine the WER or the MER. In determining WER or MER, the second transcript may be used as the ground truth such that errors are determined based on the first transcript not matching the second transcript.

At operation 350, the computer system determines an intelligibility score based on the comparison between the first and second transcripts. In some embodiments, a similarity score between the first and second transcripts may be converted into an intelligibility score, as described herein.

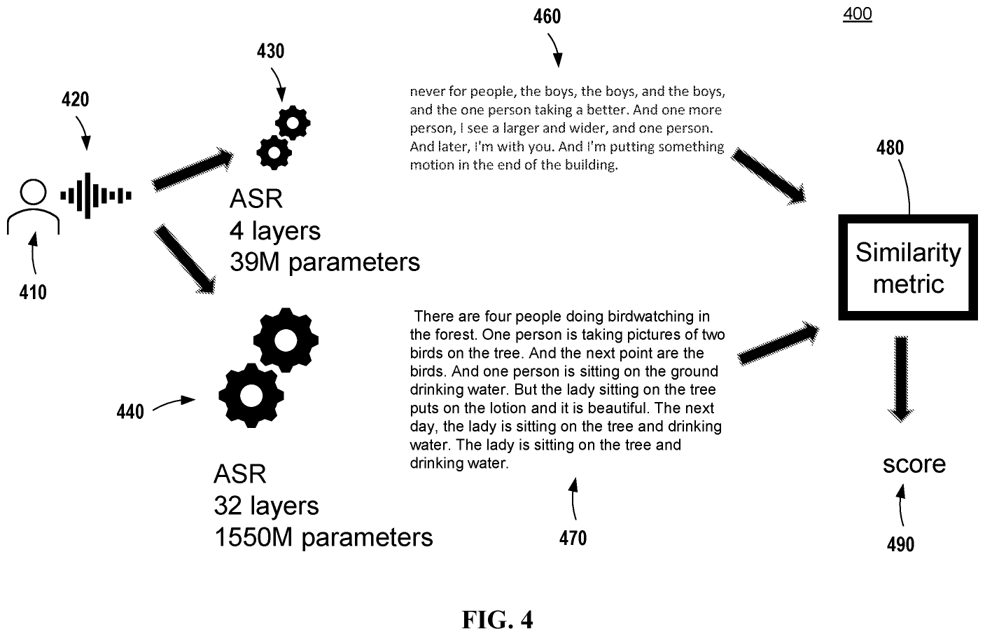

Referring now to FIG. 4, an illustrative flow diagram of intelligibility analysis according to an embodiment of the present disclosure is depicted. A speech recording 420 of a user 410 is provided to a first ASR model 430 and a second ASR model 440. ASR model 430 is smaller (4 layers and 39M parameters) and less accurate than ASR model 440 which has 32 layers and 1550M parameters. ASR model 430 processes speech recording 420 to generate transcript 460 and ASR model 440 processes speech recording 420 to generate transcript 470. Transcript 460 and transcript 470 are compared to generate a similarity metric 480. The similarity metric 480 is converted into an intelligibility score 490.

Further embodiments of the present disclosure are described in the following numbered clauses. The clauses are provided as examples and are not intended to be limiting.

Clause 1. A method for intelligibility analysis of a speech recording, the method comprising:
providing the speech recording;
generating a first transcript for the speech recording using a first automatic speech recognition (ASR) model;
providing a second transcript for the speech recording;
comparing the first transcript and the second transcript; and
determining an intelligibility score based on the comparing the first transcript and the second transcript.

Clause 2. The method of clause 1, wherein the comparing the first transcript and the second transcript includes determining a similarity metric between the first transcript and the second transcript.

Clause 3. The method of clause 2, wherein determining the intelligibility score comprises converting the similarity metric into the intelligibility score.

Clause 4. The method of clause 3, wherein converting the similarity metric is based on threshold values of the similarity metric.

Clause 5. The method of clause 4, further comprising generating the second transcript using a second ASR model to the speech recording, wherein the second ASR model is more accurate than the first ASR model.

Clause 6. The method of clause 1, further comprising generating the second transcript using a second ASR model to the speech recording, wherein the second ASR model is more accurate than the first ASR model.

Clause 7. The method of clause 1, wherein the second transcript is retrieved from an operatively coupled storage device.

Clause 8. The method of clause 1, wherein the second transcript is a script associated with the speech recording.

Clause 9. The method of clause 1, wherein the second transcript was generated by manual transcription of the speech recording.

Clause 10. A system for intelligibility analysis of a speech recording, the system comprising:
a storage device storing program instructions; and
a processor operatively connected to the storage device, the processor configured to execute the program instructions to perform operations comprising:
   providing the speech recording;
   generating a first transcript for the speech recording using a first automatic speech recognition (ASR) model;
   providing a second transcript for the speech recording;
   comparing the first transcript and the second transcript; and
   determining an intelligibility score based on the comparing the first transcript and the second transcript.

Clause 11. The system of clause 10, wherein the comparing the first transcript and the second transcript includes determining a similarity metric between the first transcript and the second transcript.

Clause 12. The system of clause 11, wherein determining the intelligibility score comprises converting the similarity metric into the intelligibility score.

Clause 13. The system of clause 12, wherein converting the similarity metric is based on threshold values of the similarity metric.

Clause 14. The system of clause 13, wherein the operations further comprise generating the second transcript using a second ASR model to the speech recording, wherein the second ASR model is more accurate than the first ASR model.

Clause 15. The system of clause 10, wherein the operations further comprise generating the second transcript using a second ASR model to the speech recording, wherein the second ASR model is more accurate than the first ASR model.

Clause 16. The system of clause 10, wherein the second transcript is retrieved from an operatively coupled storage device.

Clause 17. The system of clause 10, wherein the second transcript is a script associated with the speech recording.

Clause 18. The system of clause 10, wherein the second transcript was generated by manual transcription of the speech recording.

Clause 19. A computer program product for intelligibility analysis of a speech recording, the computer program product comprising one or more computer readable storage media having program instructions embodied therewith, the program instructions executable by a computer to cause the computer to perform operations comprising:
providing the speech recording;
generating a first transcript for the speech recording using a first automatic speech recognition (ASR) model;
providing a second transcript for the speech recording;
comparing the first transcript and the second transcript; and
determining an intelligibility score based on the comparing the first transcript and the second transcript.

Clause 20. The computer program product of clause 19, wherein the comparing the first transcript and the second transcript includes determining a similarity metric between the first transcript and the second transcript.

Clause 21. The computer program product of clause 20, wherein determining the intelligibility score comprises converting the similarity metric into the intelligibility score.

Clause 22. The computer program product of clause 21, wherein converting the similarity metric is based on threshold values of the similarity metric.

Clause 23. The computer program product of clause 22, further comprising generating the second transcript using a second ASR model to the speech recording, wherein the second ASR model is more accurate than the first ASR model.

Clause 24. The computer program product of clause 19, wherein the operations further comprise generating the second transcript using a second ASR model to the speech recording, wherein the second ASR model is more accurate than the first ASR model.

Clause 25. The computer program product of clause 19, wherein the second transcript is retrieved from an operatively coupled storage device.

Clause 26. The computer program product of clause 19, wherein the second transcript is a script associated with the speech recording.

Clause 27. The computer program product of clause 19, wherein the second transcript was generated by manual transcription of the speech recording.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A method for intelligibility analysis of a speech recording, the method comprising:
providing the speech recording;
generating a first transcript for the speech recording using a first automatic speech recognition (ASR) model;
providing a second transcript for the speech recording;
comparing the first transcript and the second transcript; and
determining an intelligibility score based on the comparing the first transcript and the second transcript.

2. The method of claim 1, wherein the comparing the first transcript and the second transcript includes determining a similarity metric between the first transcript and the second transcript.

3. The method of claim 2, wherein determining the intelligibility score comprises converting the similarity metric into the intelligibility score.

4. The method of claim 3, wherein converting the similarity metric is based on threshold values of the similarity metric.

5. The method of claim 4, further comprising generating the second transcript using a second ASR model to the speech recording, wherein the second ASR model is more accurate than the first ASR model.

6. The method of claim 1, further comprising generating the second transcript using a second ASR model to the speech recording, wherein the second ASR model is more accurate than the first ASR model.

7. The method of claim 1, wherein the second transcript is retrieved from an operatively coupled storage device.

8. The method of claim 1, wherein the second transcript is a script associated with the speech recording.

9. The method of claim 1, wherein the second transcript was generated by manual transcription of the speech recording.

10. A system for intelligibility analysis of a speech recording, the system comprising:
a storage device storing program instructions; and
a processor operatively connected to the storage device, the processor configured to execute the program instructions to perform operations comprising:
providing the speech recording;
generating a first transcript for the speech recording using a first automatic speech recognition (ASR) model;
providing a second transcript for the speech recording;
comparing the first transcript and the second transcript; and
determining an intelligibility score based on the comparing the first transcript and the second transcript.

11. The system of claim 10, wherein the comparing the first transcript and the second transcript includes determining a similarity metric between the first transcript and the second transcript.

12. The system of claim 11, wherein determining the intelligibility score comprises converting the similarity metric into the intelligibility score.

13. The system of claim 12, wherein converting the similarity metric is based on threshold values of the similarity metric.

14. The system of claim 13, wherein the operations further comprise generating the second transcript using a second ASR model to the speech recording, wherein the second ASR model is more accurate than the first ASR model.

15. The system of claim 10, wherein the operations further comprise generating the second transcript using a second ASR model to the speech recording, wherein the second ASR model is more accurate than the first ASR model.

16. A computer program product for intelligibility analysis of a speech recording, the computer program product comprising one or more computer readable storage media having program instructions embodied therewith, the program instructions executable by a computer to cause the computer to perform operations comprising:
providing the speech recording;
generating a first transcript for the speech recording using a first automatic speech recognition (ASR) model;
providing a second transcript for the speech recording;
comparing the first transcript and the second transcript; and
determining an intelligibility score based on the comparing the first transcript and the second transcript.

17. The computer program product of claim 16, wherein the comparing the first transcript and the second transcript includes determining a similarity metric between the first transcript and the second transcript.

18. The computer program product of claim 17, wherein determining the intelligibility score comprises converting the similarity metric into the intelligibility score.

19. The computer program product of claim 18, wherein converting the similarity metric is based on threshold values of the similarity metric.

20. The computer program product of claim 19, further comprising generating the second transcript using a second ASR model to the speech recording, wherein the second ASR model is more accurate than the first ASR model.
